(19) United States
(12) Patent Application Publication
(10) Pub. No.: US 2025/0229269 A1
(43) Pub. Date: Jul. 17, 2025

(54) ACOUSTIC MICROFLUIDICS ENABLED PORTABLE PCR

(71) Applicants: Transformative Biotech, LLC, Boulder, CO (US); The Regents of the University of Colorado, a body corporate, Denver, CO (US)

(72) Inventors: Xiaoyun Ding, Superior, CO (US); Robert E. Blomquist, Boulder, CO (US)

(21) Appl. No.: 19/016,825

(22) Filed: Jan. 10, 2025

Related U.S. Application Data
(60) Provisional application No. 63/619,767, filed on Jan. 11, 2024.

(51) Int. Cl. B01L 3/00

(52) U.S. Cl. CPC B01L 3/50273; B01L 2200/16; B01L 2300/0663; B01L 2300/0867; B01L 2300/12; B01L 2400/0436

(57) ABSTRACT

The invention provides methods, systems, and compositions of preparing samples for nucleic amplification by application of surface acoustic waves (SAWs).

### TECHNICAL FIELD

The present disclosure relates to systems, devices, and methods for using surface acoustic waves to prepare samples for amplifying nucleic acids.

### BACKGROUND

Nucleic acid amplification techniques, including PCR, RT-PCR, and isothermal DNA amplification, are commonly used for disease diagnosis. PCR is a common and often indispensable technique used in medical and biological research. It is a well-developed method for nucleic acid amplification in the fields of diagnosis. Based on the proper selection of specific primers, the PCR technique can be used to perform nucleic acid amplification in vitro, resulting in the production of a large quantity of a target nucleic acid sequence. PCR provides a sensitive and selective means of detecting low numbers of, or slow-growing, pathogens in clinical specimens, and hence has had considerable impact in the field of diagnostic microbiology. However, traditional PCR machines are usually bulky and relatively expensive. Besides, the PCR process is relatively time-consuming. Therefore, smaller and portable devices for preparing samples for and performing PCR processes rapidly are in crucial need.

### SUMMARY

The present invention provides systems and methods that combine the power of surface acoustic waves (SAWs), direct amplification, and integrated detection, to overcome the shortcomings of traditional PCR methods. Methods and systems of the invention provide a faster, more accessible, and reliable approach to nucleic acid analysis, particularly in the context of pathogen detection and diagnostics.

Preferred systems incorporate a microfluidic device with one or more reaction chambers designed to hold a sample and amplification reagents. Reaction chambers may be droplets in, for example, an emulsion or physical structures connected via microfluidic channels in the microfluidic system. In one embodiment, a chamber is positioned next to a substrate that is utilized to generate surface acoustic waves (SAWs). The SAWs are produced by transducers, such as interdigital transducers, that are integrated with the device's substrate. In a preferred embodiment that SAW is a piezoelectric material.

The SAWs are used to mix the sample and amplification reagents and aid in disrupting bacterial cell walls, making target nucleic acids more accessible for amplification.

Devices of the invention enable direct amplification of target nucleic acids (e.g., DNA or RNA), thus avoiding the need for any prior extraction steps. Any type of amplification can be used in the context of the invention (e.g., quantitative PCR (qPCR) or digital PCR (dPCR), and the like). Devices of the invention are designed to work with primers specific to target nucleic acids, such as those found in pathogens. Pathogens for detection include bacteria, viruses, such as SARS viruses, influenza viruses, Chlamydia trachomatis, and Neisseria gonorrhoeae.

For detection, systems of the invention incorporate an optical sensor, such as a photodiode, to analyze the signal from amplification products. Fluorescently-labeled probes, such as hydrolysis probes or quenched hairpin probes, may be included to generate a signal upon amplification. This signal is then processed by a controller device, which is configured to receive and analyze the output from the optical sensor.

In addition to the elements described above, systems of the invention are designed for a variety of sample types, including saliva, respiratory mucosa (e.g., from nasal or throat swabs), blood, urine, cerebrospinal fluid, pus, stool, and genital secretions. Sample loading preferably involves introducing both the sample and the amplification reagents, as well as a signal reporter, directly into the reaction volume.

To prepare samples, systems use a buffer that simplifies the process by inactivating viral particles and stabilizing the released nucleic acids for amplification without requiring an extraction step. Additionally, the device can be used to perform a heat inactivation step at 95° C. for 5 minutes, further ensuring that viral particles are inactivated.

In certain embodiments, the transducers are interdigital transducers (IDTs). The IDTs generate surface acoustic waves (SAWs) that propagate on the surface of the substrate towards the chamber. SAWs that are generated from the IDT will induce acoustic streaming in the chamber and achieve a fast mixing of the sample and amplification reagents in a very short time. Such fast mixing in low Reynolds number plays in important role in portable PCR. Due to the small channel dimensions at this scale, mixing by diffusion alone is slow and ineffective. SAWs provide a mechanism for quickly mixing the sample and reagents. SAWs that are generated from the IDT will further increase the temperature rapidly in the chamber through acoustic heating effect. Such fast heating is required for the following PCR process. Nucleic acids and reagents will be mixed and heated for nucleic acid amplification in the chamber in a short amount of time, typically less than 30 minutes. In certain embodiments, amplification products are detected using detectably labeled probes. In particular embodiments, the detectably labeled probes are optically labeled probes, such as fluorescently labeled probes. In certain embodiments, the loading step further comprises introducing, into the reaction volume, a reporter that gives a signal from amplification products. In certain embodiments, the method further comprises amplifying the nucleic acids in the sample to form the amplification products.

The present disclosure provides methods of sample preparation for extraction-free nucleic acid analysis involving loading a sample and amplification reagents into a reaction volume on a microfluidic system, wherein the sample comprises a target nucleic acid; and mixing the sample with the amplification reagents in the reaction volume by application of surface acoustic waves.

In certain embodiments, the methods of the present invention further comprise detecting the signal from the amplification products with an optical sensor. After nucleic acid amplification e.g., from PCR, the fluorescent information (e.g., from the signal from the amplification products) may be measured by an optical sensor such as a photodiode underneath the substrate. The signals from the optical sensor may be collected and analyzed by a controller device, immediately. In certain embodiments, the controller device is a smart device comprising a power source.

In certain embodiments, systems of the present disclosure involve systems for preparing nucleic acids for amplification, the systems comprising a substrate comprising a reaction chamber and one or more transducers connected to the substrate and operable to apply surface acoustic waves that promote mixing of nucleic acid with amplification reagents to the reaction volume. The system may comprise two interdigital transducers. The system may comprise a channel comprising three inlets. The system may further comprise a first inlet configured to load a sample, and a second inlet and a third inlet that are configured to load a chemical reagent. The system may further comprise at least one interdigital transducer operable to generate surface acoustic waves that propagate on the surface of the substrate towards the chamber. The system may further comprise an optical sensor. The optical sensor may be a photodiode. They system may further comprise a controller device. In certain embodiments, the controller device is a smart device comprising a power source. The controller device may further receive and analyze output of the optical sensor. The system may further comprise a controller device that includes a power source such as a battery. The controller device may comprise a computerized device such as a desktop or portable computer, a smart device (e.g., a smart phone or tablet computer), executing one or more software programs to receive the signals from the optical sensor and provide, for example an assessment of the signals from the optical sensor.

This integrated microfluidic system brings together SAWs, direct amplification, and integrated detection, into a compact and efficient device for rapid nucleic acid analysis, offering a more accessible faster and reliable approach to pathogen detection.

### BRIEF DESCRIPTION OF THE DRAWINGS

FIG. 1. Shows an exemplary schematic of a system for using surface acoustic waves to prepare samples for amplifying nucleic acids.

FIG. 2. Shows an exemplary side view of a system for using surface acoustic waves to prepare samples for amplifying nucleic acids.

FIG. 3. Shows an exemplary top view of a system for using surface acoustic waves to prepare samples for amplifying nucleic acids.

### DETAILED DESCRIPTION

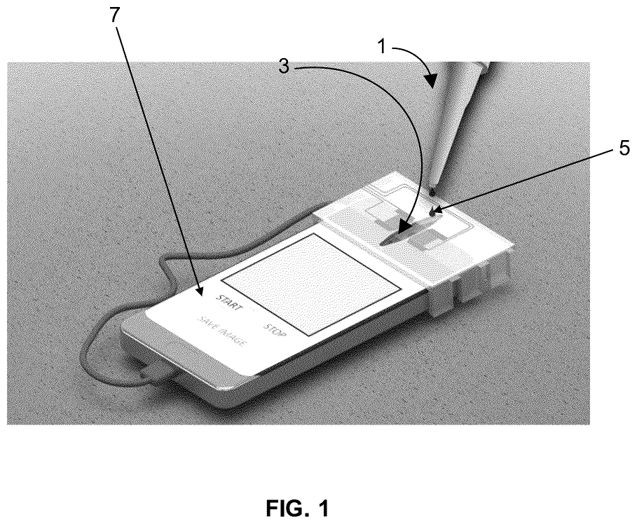

FIG. 1 shows an exemplary system for preparing samples comprising loading 1 into a reaction volume 3, a sample in a droplet of an emulsion 5 comprising a template nucleic acid and amplification reagents. The system further comprises a controller device 7.

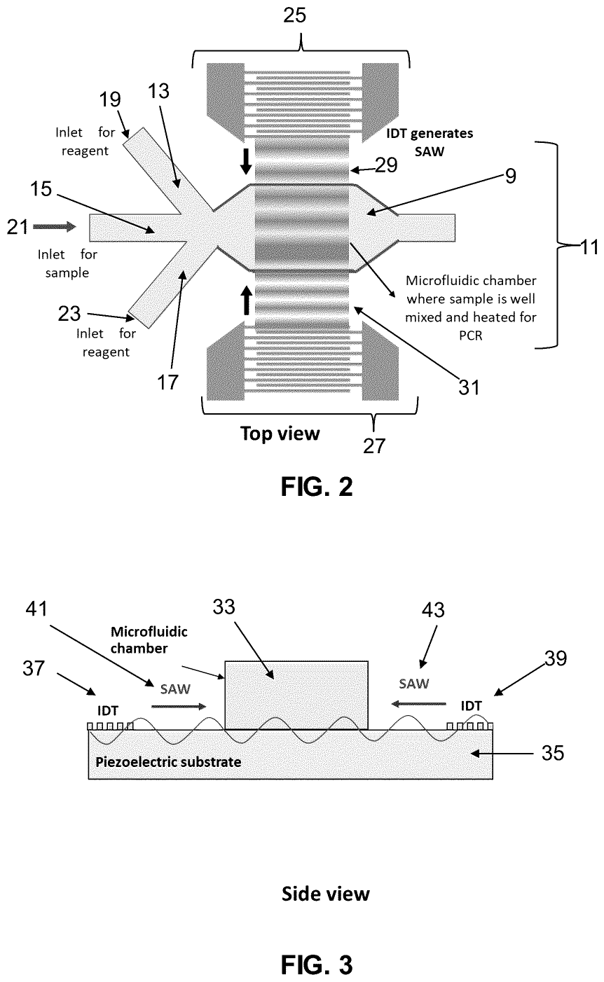

FIG. 2 shows a top view of an exemplary system for preparing samples comprising a reaction volume (e.g., a chamber) 9 on a microfluidic system 11 connected to channels 13, 15, and 17. The channels comprise inlets 19, 21, and 23 for loading reagents and/or samples. FIG. 2 further shows interdigital transducers (IDTs) 25 and 27. Surface acoustic waves (SAWs) 29 and 31 generated by the transducers propagate on the surface of the substrate towards the chamber 9.

FIG. 3. shows a side view of an exemplary system for preparing samples comprising a reaction volume (e.g., a chamber) 33 adjacent to a piezoelectric substrate 35, wherein the substrate comprises two interdigital transducers 37 and 39 operable to transmit surface acoustic waves 41 and 43 across the substrate 35 into the reaction volume (e.g., a chamber) 33.

Systems of the invention for extraction-free nucleic acid analysis integrates SAWs, direct amplification, and integrated detection within a compact and portable device. Preferred systems utilize a microfluidic substrate, typically made of a piezoelectric material, which houses reaction chambers and integrated components. Reaction chambers are designed to hold the sample and amplification reagents and can be configured in an aqueous phase (e.g., a droplet) or a chamber connected to one or more channels within the system. The chamber is positioned adjacent to the substrate for efficient interaction with SAWs. Transducers, such as interdigital transducers (IDTs), are integrated with the substrate and generate SAWs, which propagate across the substrate into the reaction volume, promising mixing and disruption of cell walls.

The SAWs perform a dual function: they facilitate the mixing of the sample with amplification reagents and induce mechanical stress on bacterial cell walls, disrupting them to make the target nucleic acids accessible for amplification without extraction. Systems described herein support a variety of sample types including saliva, respiratory mucosa, vaginal and rectal swabs, blood, urine, cerebrospinal fluid, pus, stool, and genital secretions.

In a preferred method, samples are mixed with a buffer that includes nuclease-free water, an antifungal agent like Amphotericin B, an antibiotic agent like Penicillin and Streptomycin, a ribonuclease inhibitor, and a reducing agent like Tris(2-carboxyethyl) phosphine hydrochloride. The buffer is substantially free of PCR inhibitory substances. Following the mixing with the buffer, samples may be heat-inactivated at 95° Celsius for 5 minutes.

Inventive systems allow for the direct amplification of target nucleic acids (e.g., DNA or RNA) without prior extraction, using primers specific to the target sequences. Amplification can be achieved through qPCR or dPCR. The source for target nucleic acids can be bacteria or viruses, including those associated with infectious pathogens, such as SARS-COV-2, influenza viruses, Chlamydia trachomatis, and Neisseria gonorrhoeae. For detection, the systems incorporate an optical sensor, often a photodiode, to analyze the signal from the amplification products. Fluorescently labeled probes, such as hydrolysis probes or quenched hairpin probes, may be used as reporters. The optical sensor is connected to a controller device with its own power source, which is configured to receive and analyze output from the optical sensor. A power source is connected to the transducers to enable the application of SAWs.

SAWs are generated by transducers on a substrate (e.g., a piezoelectric substrate), and offer a contactless means of interacting with fluids and cells at the microscale. The application of SAWs in this system is central to achieving extraction-free nucleic acid analysis.

The SAWs are generated by applying an electrical signal to interdigital transducers (IDTs) which are fabricated on a substrate (e.g., a piezoelectric substrate). The piezoelectric material, when subjected to an electrical field, deforms and generates mechanical waves that propagate along the surface of the substrate. This precise generation of waves allows for fine control over their properties, such as frequency, amplitude, and direction.

Once generated, the SAWs travel across the substrate and, upon encountering the reaction volume containing the sample and amplification reagents, they create a variety of physical effects. The SAWs induce acoustic streaming within the fluid, resulting in rapid and efficient mixing of the sample with the amplification reagents. Unlike conventional mechanical mixing methods, SAWs provide a contactless approach, reducing the risk of contamination and system wear. When saws interact with biological samples containing cells, such as bacteria, they exert mechanical stress on the cell walls. This stress causes the cell walls to rupture, releasing the intracellular contents, including the target nucleic acids. This cell disruption allows access to the nucleic acids without requiring traditional extraction methods.

Systems of the invention are designed to accommodate a wide range of biological samples, including but not limited to: saliva, respiratory mucosa, vaginal and rectal swabs, blood, urine, cerebrospinal fluid, pus, stool, and genital secretions. These samples can be collected using conventional methods and are introduced into the microfluidic system for processing. The selection of sample type depends on the specific diagnostic target. For example, saliva or respiratory mucosa may be preferred for detecting respiratory pathogens. Samples can be self-collected, at home without supervision from a healthcare professional, into a buffer, simplifying the collection process.

Samples are collected in a buffer that is tailored to prepare nucleic acids for direct amplification. The buffer includes nuclease-free water to main the integrity of nucleic acids, antifungal agent, such as Amphotericin B, to prevent fungal contamination, antibiotic agent such as penicillin and streptomycin to prevent bacterial growth, ribonuclease inhibitor to protect RNA from degradation, and a reducing agent such as Tris(2-carboxyethyl) phosphine hydrochloride to stabilize the sample and enhance proteinase K activity.

A sample is loaded into a reaction volume on the microfluidic system along with the amplification reagents. SAWs are then used to mix the sample with the amplification reagents. The samples are prepared for analysis without the need for traditional nucleic acid extraction methods. Instead, the buffer and SAWs act to release the nucleic acids from the sample. Samples mixed with the buffer can be heat-inactivated at 95° Celsius for 5 minutes. This step helps to inactivate any pathogens in the sample.

Following the loading step, the target nucleic acids within the sample are amplified directly within the reaction volume using specific primers. The amplification can be achieved via qPCR or dPCR. After amplification, the system uses a reporter, such as a fluorescently labeled probe, to detect presence of amplified target nucleic acids. This probe can be a hydrolysis probe or a quenched hairpin probe. An optical sensor, often a photodiode, is used to detect the signal from the amplification products.

Biological samples, such as saliva, can be collected directly into a container with the buffer, ready for processing. Swabs, such as nasal or vaginal swabs, are placed into a buffer for collection and preservation. Nasal swabs may be combined with saliva samples to increase the amount of viral material available for detection. Samples, once mixed with the buffer and heat-inactivated, are ready for amplification and detection steps in the microfluidic system.

The system's use of SAWs to disrupt cell walls is particularly relevant for bacterial pathogens. Gram-Positive Bacteria have a thick peptidoglycan layer in their cell wall. The SAWs in the microfluidic system can effectively disrupt this layer, enabling access to the bacterial nucleic acids for amplification. Gram-Negative bacteria have a thinner peptidoglycan layer, surrounded by an outer membrane containing lipopolysaccharides (LPS). While the peptidoglycan layer is not as thick as in Gram-positive bacteria, the outer membrane provides an additional barrier. The mechanical stress from SAWs is able to overcome the structure to facilitate lysis and access to nucleic acids.

The buffers as used herein may refer to a mixture of components that inactivate the sample and make the nucleic acid available for amplification. The buffers are designed to facilitate the direct use of nucleic acids for downstream PCR without prior extraction. A component of the buffer is a reducing agent, specifically Tris(2-carboxyethyl) phosphine hydrochloride (TCEP). TCEP helps to reduce disulfide bonds and prevent the formation of aggregates. To prevent microbial growth and contamination, the buffers may include an antifungal agent and an antibiotic agent. The antifungal agent may be Amphotericin B, and the antibiotic agent may be Penicillin and/or Streptomycin. A ribonuclease inhibitor may also be included to protect RNA from degradation by ribonucleases, which are enzymes that can break down RNA molecules. The buffers may be prepared using nuclease-free water to avoid introducing any nucleases that could degrade the sample's nucleic acids.

For saliva samples, the buffer may comprise nuclease-free water, TCEP, a ribonuclease inhibitor, Amphotericin B, and Penicillin and/or Streptomycin. In some embodiments, the buffer also includes a protease, such as Proteinase K, to aid in the breakdown of proteins in the sample.

For swab samples, the buffer may comprise nuclease-free water, Tris-Borate-EDTA (TBE), a ribonuclease inhibitor, Amphotericin B, and Penicillin and/or Streptomycin.

The buffer is substantially free of PCR inhibitory substances, allowing the PCR reaction to occur effectively without interference from the buffer components. The buffers are designed to allow direct sample testing without the need for nucleic acid extraction.

The invention relates to methods of preparing samples for amplification. In certain embodiments, methods of the invention involve loading, into a reaction volume on a microfluidic system, a sample comprising a template nucleic acid and amplification reagents and mixing the template nucleic acid with the amplification reagents in the reaction volume by application of surface acoustic waves (SAWs). A surface acoustic wave (SAW) is an acoustic wave traveling along the surface of a material exhibiting elasticity. In certain embodiments, the reaction volume comprises a chamber connected to at least one channel on a microfluidic system. In certain other embodiments, the channel on the microfluidic system comprises at least one inlet. The inlets may be used to load in samples and/or chemical reagents that are necessary for subsequent reactions or tests (e.g. nucleic acid amplification).

In one embodiment, the method involves loading a sample and amplification reagents into a reaction volume situated on a microfluidic system. The reaction volume can be configured in several ways. In some embodiments, the reaction volume may be in the form of a droplet. This droplet can be a aqueous solution, but it can also be a droplet of an emulsion. An emulsion is a mixture of two or more liquids that are normally immiscible, such as oil and water. The emulsion can be a water-in-oil or oil-in-water type. The use of a droplet of an emulsion can help compartmentalize the reaction mixture, providing a microenvironment for amplification and analysis. This compartmentalization can be useful in dPCR, as it allows for the quantification of target nucleic acids at single-molecule level.

In other embodiments, the reaction volume takes the form of a chamber. This chamber is connected to at least one channel on the microfluidic system. This channel can be used to deliver the sample and amplification reagents to the chamber, as well as remove the amplicons after amplification. The chamber itself can be designed with various features to optimize the reaction, such as specific dimensions and surface properties. The chamber may be adjacent to a substrate. The substrate can serve as a base for the chamber and can also incorporate components that facilitate the reaction.

In certain embodiments, the chamber is adjacent to a substrate. In certain embodiments, the substrate comprises one or more transducers operable to transmit surface acoustic waves across the substrate into the reaction volume. In certain embodiments, the transducers comprise interdigital transducers and the substrate comprises a piezoelectric material such as lithium niobate. A transducer converts a signal in one form of energy to a signal in another. Transducers are utilized at the boundaries of automation, measurement, and control systems, where electrical signals are converted to and from other physical quantities (e.g., energy, force, torque, light, motion, position, etc.). An interdigital transducer (IDT) is a device that consists of two interlocking arrays of metallic electrodes. These metallic electrodes are deposited on the surface of a piezoelectric substrate, such as quartz or lithium niobate, to form a periodic structure. IDTs convert electric signals to surface acoustic waves (SAWs) by generating periodically distributed mechanical forces via piezoelectric effect. The surface acoustic waves (SAWs) generated by the transducers propagate on the surface of the substrate towards the chamber. A surface acoustic wave (SAW) is an acoustic wave traveling along the surface of a material exhibiting elasticity. SAWs that are generated from the transducers will induce acoustic streaming in the chamber and achieve a fast mixing of the sample media in a very short time. Such fast mixing in low Reynolds number is a critical step for portable PCR. SAWs that are generated from the transducers will also increase the temperature rapidly in the chamber through acoustic heating effect. Such fast heating is required for the following reactions e.g., for nucleic amplification. Samples and reagents will be mixed and heated for PCR in the microfluidic chamber in a short amount of time, e.g., less than 30 minutes. In certain embodiments, the method further comprises amplifying the nucleic acid (e.g. PCR) to form amplification products. In certain embodiments, the method further comprises detecting the signal from the amplification products with an optical sensor. In certain embodiments, the optical sensor is a photodiode.

Optical sensors are powerful analytic tools capable of providing analyte information remotely. Typically, optical sensors for chemical or biological molecules are composed of molecular recognition elements and signal transducers. The molecular recognition elements interact with the target analytes under study and provide information about their presence, concentration, and other physical properties of interest. When target molecules enter the system, the sensors produce detectable changes in their signals-which are then transduced into easily measured and quantified optical signals. In certain embodiments, the optical sensor is a photodiode. A photodiode is a semiconductor diode sensitive to photon radiation, such as visible light, infrared or ultraviolet radiation, X-rays and gamma rays.

In certain embodiments, the sample comprises template nucleic acids and amplification reagents. Nucleic acids are large biomolecules that are crucial in all cells and viruses. They are composed of nucleotides, which are the monomer components: a 5-carbon sugar, a phosphate group and a nitrogenous base. The two main classes of nucleic acids are deoxyribonucleic acid (DNA) and ribonucleic acid (RNA). If the sugar is ribose, the polymer is RNA; if the sugar is deoxyribose, a variant of ribose, the polymer is DNA.

PCR amplifies a specific region of a DNA strand. Most PCR methods amplify DNA fragments of between 0.1 and 10 kilo base pairs (kbp) in length. In certain embodiments, the PCR amplifies fragments up to 40 kbp. A basic PCR set-up requires several components and amplification reagents, including a DNA template that contains the DNA target region to amplify, a DNA polymerase, an enzyme that polymerizes new DNA strands (e.g., heat-resistant Taq polymerase), two DNA primers that are complementary to 3′ (three prime) ends of each of the sense and anti-sense strands of the DNA target, deoxynucleoside triphosphates, or dNTPs, a buffer providing a suitable chemical environment for optimum activity and stability of the DNA polymerase, bivalent cations (e.g., magnesium (Mg) or manganese (Mn) ions e.g., Mg2+ and Mn2+) and monovalent cations, e.g., potassium (K) ions.

PCR relies on highly specific and efficient amplification of the target sequence and the inclusion of reporters that enable the quantification of PCR products. Reporters can be DNA-binding dyes such as SYBR® Green or fluorescently labeled PCR primers or probes. There are many different kinds of fluorescent dyes, PCR primers, and probes.

In certain embodiments, the loading step further comprises introducing, into the reaction volume, a reporter that gives a signal from amplification products. In certain embodiments, the reporter comprises a fluorescent probe. In certain embodiments, the fluorescent probe is a hydrolysis probe or a quenched hairpin probe. In certain embodiments, amplified products are detected using detectably labeled probes. In particular embodiments, the detectably labeled probes are optically labeled probes, such as fluorescently labeled probes. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl] naphthalimide-3,5 disulfonate; N-(4-anilino-1-naphthyl) maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron. TM. Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. Preferred fluorescent labels are FAM and VICTM (from Applied Biosystems). Labels other than fluorescent labels are contemplated by the invention, including other optically-detectable labels.

The invention further relates to a system for preparing nucleic acids for amplification, the system comprising a substrate comprising a reaction chamber and one or more transducers connected to the substrate and operable to apply surface acoustic waves that promote mixing of nucleic acid with amplification reagents to the reaction volume. The substrate may further comprise piezoelectric material. The reaction chamber may be connected to at least one channel comprising at least one inlet. The system may further comprise an optical sensor. The optical sensor is a photodiode. The system may further comprise a power source connected to the one or more transducers. The system may further comprise a controller device operable to receive and analyze output of the optical sensor. The system may further comprise a controller device comprising a power source. The system may further comprise a controller device that includes a power source such as a battery. The controller device may comprise a computerized device such as a desktop or portable computer, a smart phone or tablet computer, executing one or more software programs to receive the sensor data and provide, for example an assessment of the signals from the optical sensor.

The system may include a controller device comprising a processor and a memory and which may be coupled to the optical sensor arrangement. The controller includes the hardware and the firmware that, with the support of program memory and random access memory controls one or more sensors, receives the sensor data, processes the sensor data, stores the processed data in memory, communicates with a transmitter or directly to a database server system via a communication device, and manages the power source. The memory may store the processed data until successful transmission to the data to the database server is confirmed.

The controller device may be operable to receive signal data corresponding to received light generated by the optical detector. The controller may estimate total particle concentration data and concentration data using the signal data. The system may further comprise an electronic communication device (e.g., wireless transmitter, transceiver) and a power source (e.g., battery) coupled to the controller. The controller device may be connected to each sensor for power supply to the sensor and data output from each sensor to the control device (e.g. signals from the optical sensor). The sensors may be connected to a printed circuit board (PCB). A controller device (e.g., microcontroller, firmware, memory storage module, wireless transmission module) are also connected to the PCB. For local power supply, a battery is connected to the PCB, as well as a charging and/or communication port. A power charging port may be controlled by the controller to charge the local power supply. The power source provides power to system. For example, the power source is a battery. The system itself may be plugged into an outlet power source (e.g., wall power outlet, power provided by a USB connection, etc.). For example, a USB port may also act as a connection to an external power source or wired communication channel to access the data. The controller device may be connected to the battery and the charging port via the PCB.

The processor may be any suitable processing device configured to run and/or execute a set of instructions or code and may include one or more data processors, image processors, graphics processing units, physics processing units, digital signal processors, and/or central processing units. The processor may be, for example, a general-purpose processor, Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types, including but not limited to metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures mixed analog and digital, and the like.

In some variations, the memory may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, and the like. As used herein, database refers to a data storage resource. The memory may store instructions to cause the processor (to execute modules, processes and/or functions associated with the computing device, such as valve control, signal data processing, data analysis, sensor control, communication, and/or user settings. In some variations, storage may be network-based and accessible for one or more authorized users. Network-based storage may be referred to as remote data storage or cloud data storage. EMG signal data stored in cloud data storage (e.g., database) may be accessible to respective users via a network, such as the Internet. In some variations, the database server may be a cloud-based FPGA.

The systems, devices, and/or methods described herein may be performed by software (executed on hardware), hardware (e.g., a controller device), or a combination thereof. Modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

The database server system includes network connectivity and communication, data processing via a computing processor and random-access memory, and storage capabilities via hard drives or similar hardware. The database server system may include an application program interface (API) to define the communication procedures and protocols for interfacing to the hardware patient monitoring device, transmitter module, and/or the user interface software system. In addition, the database server system may have an API for communication with external systems for the purpose of storing additional data metrics for monitoring trends, alert generation, diagnosis or alternate purposes.

A database server system may be comprised of cloud-based storage and/or local servers. Network-based storage may be referred to as remote data storage or cloud data storage. EMG signal data stored in cloud data storage (e.g., database) may be accessible to respective users via a network, such as the Internet. Data stored on a cloud database may be accessible from any account and/or device that is granted access to that data.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of sample preparation for extraction-free nucleic acid analysis, the method comprising:
loading a sample and amplification reagents into a reaction volume on a microfluidic system, wherein the sample comprises a target nucleic acid; and
mixing the sample with the amplification reagents in the reaction volume by application of surface acoustic waves.

2. The method of claim 1, further comprising:
amplifying the target nucleic acid to produce amplicons within the reaction volume without prior extraction of the target nucleic acid; and
analyzing the amplicons to detect presence of a target nucleic acid.

3. The method of claim 1, wherein the sample is selected from the group comprising: saliva, respiratory mucosa, nasal swabs, vaginal swabs, rectal swabs, blood, urine, cerebrospinal fluid, pus, stool, and genital secretions.

4. The method of claim 1, wherein the surface acoustic waves facilitate disruption of bacterial cell walls within the sample to make the target nucleic acid accessible for amplification.

5. The method of claim 1, wherein the microfluidic system comprises one or multiple reaction chambers, each configured for the amplification of specific probes for specific target nucleic acids.

6. The method of claim 5, wherein the specific target nucleic acids are associated with an infectious pathogen.

7. The method of claim 6, wherein the infectious pathogen is selected from the group comprising Severe acute respiratory syndrome coronavirus, influenza virus, Mycobacterium tuberculosis, Chlamydia trachomatis, and Neisseria gonorrhoeae.

8. The method of claim 2, wherein the amplifying step comprises quantitative PCR (qPCR) or digital PCR (dPCR).

9. The method of claim 2, further comprising quantifying the target nucleic acid.

10. The method of claim 2, wherein the analyzing step comprises sequencing the amplicons.

11. The method of claim 1, wherein the sample is self-collected in a buffer.

12. The method of claim 11, wherein the buffer comprises nuclease-free water, an antifungal agent, an antibiotic agent, a ribonuclease inhibitor, and a reducing agent.

13. The method of claim 12, wherein the reducing agent is Tris(2-carboxyethyl) phosphine hydrochloride.

14. The method of claim 12, wherein the antifungal agent comprises Amphotericin B and the antibiotic agent comprises Penicillin and Streptomycin.

15. The method of claim 12, further comprising heating the self-collected sample in the buffer at 95° C. for 5 minutes.

16. The method of claim 11, wherein the buffer is substantially free of PCR inhibitory substances.

17. The method of claim 1, wherein the reaction volume comprises a droplet of an emulsion.

18. The method of claim 1, wherein the reaction volume is a chamber connected to at least one channel on the microfluidic system.

19. The method of claim 3, wherein the chamber is adjacent to a substrate.

20. The method of claim 4, wherein the substrate comprises one or more transducers operable to transmit surface acoustic waves across the substrate into the reaction volume.

21. The method of claim 5, wherein the transducers comprise interdigital transducers and the substrate comprises a piezoelectric material.

22. The method of claim 1, wherein the loading step further comprises introducing, into the reaction volume, a reporter that gives a signal from amplification products.

23. The method of claim 7, wherein the reporter comprises a fluorescently labeled probe.

24. The method of claim 8, wherein the fluorescent labeled probe is a hydrolysis probe or a quenched hairpin probe.

25. The method of claim 2, wherein the analyzing step comprises detecting the signal from the amplification products with an optical sensor.

26. The method of claim 25, wherein the optical sensor is a photodiode.

27. A system for preparing nucleic acids for amplification, the system comprising:
a substrate comprising a reaction chamber; and
one or more transducers connected to the substrate and operable to apply surface acoustic waves that promote mixing of nucleic acid with amplification reagents to the reaction volume.

28. The system of claim 27, wherein the substrate comprises piezoelectric material.

29. The system of claim 27, wherein the reaction chamber is connected to at least one channel comprising at least one inlet.

30. The system of claim 29, further comprising an optical sensor.

31. The system of claim 30, wherein the optical sensor is a photodiode.

32. The system of claim 29, further comprising a power source connected to the one or more transducers.

33. The system of claim 30, further comprising a controller device operable to receive and analyze output of the optical sensor.

34. The system of claim 33, wherein the controller device comprises a power source.
